SEQUENCE LISTING (5 sequences)

SEQ ID NO: 1 - DNA, 57 bp, Artificial sequence
  beta-catenin-antisense promoter-linked primer
ccagtgaattgtaatacgactcactataggcgctctgaagacagtctgtcgtgatgg

SEQ ID NO: 2 - DNA, 24 bp, Artificial sequence
  beta-catenin-sense primer
atggcaacccaagctgacttgatc

SEQ ID NO: 3 - DNA, 24 bp, Artificial sequence
  A sense primer for construction of a RNA/DNA hybrid targeting HIV
ggatgncngncnccttgttggtcc

SEQ ID NO: 4 - DNA, 80 bp, Artificial sequence
  Partial Mus musculus tyrosinase (tyr) sense DNA (sDNA) sequence
gtgctcaggcaacttcatgggtttcaactgcggaaactgtaagtttggatttgggggcccaaattgtacagagaagcgag

SEQ ID NO: 5 - RNA, 80 nt, Artificial sequence
  complementary antisense RNA (aRNA) to SEQ ID.4
cucgcuucucuguacaauuugggcccccaaauccaaacuuacaguuuccgcaguugaaacccaugaaguugccugagcac

(19) United States
(12) Patent Application Publication
(10) Pub. No.: US 2003/0104401 A1
(43) Pub. Date: Jun. 5, 2003

(54) Gene silencing using sense DNA and antisense RNA hybrid constructs

(75) Inventor: Shi-Lung Lin, Alhambra, CA (US)

Correspondence Address: Raymond Y. CHAN, 108 N. YNEZ AVE. #128, MONTERY PARK, CA, 91754, US

(73) Assignee: Epiclone, Inc.

(21) Appl. No.: 10/052,486

(22) Filed: Jan. 18, 2002

Related U.S. Application Data
(60) Provisional application No. 60/351,183, filed on Nov. 12, 2001.

(51) Int. Cl. C12Q 1/68; C12P 19/34; C12N 15/88; C12N 5/02

(52) U.S. Cl. 435/6; 435/91.2; 435/375; 435/456; 435/458

(57) ABSTRACT

The present invention provides a stable and efficient method for stimulating RNAi-related gene silencing effects. The present invention also provides a fast, simple and specific method for generating amplified cDNA-aRNA hybrids whose quantity and quality are high enough to be used in specific gene silencing transfection. This improved RNA-polymerase cycling reaction (RNA-PCR) relies upon a thermocycling procedure of in-vitro transcription and reverse transcription to bring up the amount of DNA-RNA hybrids up to two thousand folds within one round of the reaction. The resulting cDNA-aRNA product is useful for silencing either endogenous or exogenous gene expression in transfected cells.

### CLAIM OF PRIORITY

The present application claims priority to U.S. Provisional Application Serial No. (to be assigned) (Attorney Docket 269/157) filed on Nov. 12, 2001, entitled GENE SILENCING USING SENSE DNA AND ANTISENSE RNA HYBRID CONSTRUCTS, which is hereby incorporated by reference as if fully set forth herein.

### BACKGROUND OF THE INVENTION

1. Field of The Invention

The present invention generally relates to the field of methods for generating DNA-RNA hybrids for gene knockout transfection in vitro and in vivo. More particularly, the present invention relates to gene silencing using sense cDNA-antisense RNA hybrids and methods for generating such cDNA-aRNA hybrids for silencing intracellular gene expression.

2. Description of The Prior Art

The following references are pertinent to this invention:

1. Grant et.al., “Dissecting the mechanisms of posttranscriptional gene silencing: divide and conquer”, Cell 96, 303-306 (1999).

2. Kennerdell, J. R. and Carthew, R. M., Cell 95, 1017-1026 (1998).

3. Misquitta, L. and Paterson, B. M. (1999) Proc. Natl. Acad. Sci. USA 96, 1451-1456.

4. Pal-Bhadra, M., Bhadra, U., and Birchler, J. A. (1999) Cell 99, 35-46.

5. Tabara, H., Sarkissian, M., Kelly, W. G., Fleenor, J., Grishok, A., and Timmons, L. (1999) Cell 99, 123-132.

6. Ketting, R. F., Haverkamp, T. H., van Luenen, H. G., and Plasterk, R. H. (1999) Cell 99, 133-141.

7. Fire, A., Xu, S., Montgomery, M. K., Kostas, S. A., Driver, S. E., and Mello, C. C., “Potent and specific genetic interference by double-stranded RNA in Caenorhabditis elegans”, Nature 391: 806-811 (1998).

8. Grishok, A., Tabara, H., and Mello, C. C. (2000) Science 287, 2494-2497.

9. Wargelius, A., Ellingsen, S., and Fjose, A. (1999) Biochem. Biophys. Res. Commun. 263, 156-161.

10. Wianny, F. and Zernicka-Goetz, M. (2000) Nature Cell Biol. 2, 70-75.

11. Bosher, J. M. and Labouesse, M., “RNA interference: genetic wand and genetic watchdog”, Nature Cell Biology 2: 31-36 (2000).

12. Zamore, P. D., Tuschl, T., Sharp, P. A., and Bartel, D. P. (2000) Cell 101, 25-33.

13. Yang, D., Lu, H., and Erickson, J. W. (2000) Current Biology 10, 1191-1200.

14. Cogoni, C. and Macino, G. (1999) Nature 399, 166-169.

15. Smardon, A., Spoerke, J. M., Stacey, S. C., Klein, M. E., Mackin, N., and Maine, E. M. (2000) Curr. Biol. 10, 169-171.

16. Raffo, A. J., Perlman, H., Chen, M. W., Day, M. L., Streitman, J. S., and Buttyan, R. (1995) Cancer Res. 55, 4438-4445.

17. Colombel, M., Symmans, F., Gil, S., O'Toole, K. M., Chopin, D., Benson, M., Olsson, C. A., Korsmeyer, S., Buttyan, R.(1993) Am. J. Pathol. 143, 390-400.

18. Berchem, G. J., Bosseler, M., Sugars, L. Y., Voeller, H. J., Zeitlin, S., and Gelmann, E. P. (1995) Cancer Res. 55, 735-738.

19. McConkey, D. J., Greene, G., and Pettaway, C. A. (1996) Cancer Res. 56, 5594-5599.

20. Elbashir et.al., “Duplexes of 21-nucleotide RNAs mediate RNA interference in cultured mammalian cells”, Nature 411, 494-498 (2001).

21. Stark et.al., “How cells respond to interferons”, Annu. Rev. Biochem. 67, 227-264 (1998).

22. Bahramian et.al., “Transcriptional and posttranscriptional silencing of rodent α1(I) collagen by homologous transcriptionally self-silenced transgene”, Mol. Cell. Biology 19, 274-283 (1999).

23. Shi-Lung Lin, Cheng-Ming Chuong, Randall B. Widelitz and Shao-Yao Ying, “In vivo analysis of cancerous gene expression by RNA-polymerase chain reaction”, Nucleic Acid Res. 27, 4585-4589 (1999).

24. Alexeev et.al., “Localized in vivo genotypic and phenotypic correction of the albino mutation in skin by RNA-DNA oligonucleotide”, Nat. Biotechnol. 18, 43-47 (2000).

25. Shi-Lung Lin, Cheng-Ming Chuong and Shao-Yao Ying, “A novel mRNA-cDNA interference phenomenon for silencing bcl-2 expression in human LNCaP cells”, Biochem. Biophys. Res. Commun. 281, 639-644 (2001).

26. Shi-Lung Lin and Shao-Yao Ying, “D-RNAi (Messenger RNA-antisense DNA Interference Phenomenon) is a novel defense system against cancers and viral infections”, Current Cancer Drug Targets in press (2001).

27. Sambrook et.al., “Molecular Cloning, 2^{nd} Ed.”, Cold Spring Harbor Laboratory Press, pp8.11-8.19 and pp 7.39-7.52 (1989).

28. Compton, J., “Nucleic acid sequence-based amplification”, Nature 350, 91-92 (1991).

29. Kacian et al., Proc. Natl. Acad. Sci. USA 69, 3038-3044 (1972).

30. Chamberlin et al., Nature 228, 227-231 (1970).

31. Myers and Gelfand, Biochemistry 30, 7662-7666 (1991).

32. Scott W. Knight and Brenda L. Bass, “A role for the RNase III enzyme DCR-1 in RNA interference and germ line development in Caenorhabditis elegans”, Science 293, 2269-2271 (2001).

33. Kwoh et al., Proc. Natl. Acad. Sci. USA 86, 1173-1177 (1989).

34. Embleton et al., Nucl. Acids Res. 20, 3831-3837 (1992).

35. U.S. Pat. No. 4,683,195 issued to Mullis et.al.

36. U.S. Pat. No. 4,683,202 issued to Mullis et.al.

37. U.S. Pat. No. 4,965,188 issued to Mullis et.al.

38. U.S. Pat. No. 5,075,216 issued to Innis et.al.

39. U.S. Pat. No. 5,322,770 issued to Gelfand et.al.

40. U.S. Pat. No. 5,817,465 issued to Mallet et.al.

41. U.S. Pat. No. 5,888,779 issued to Kacian et.al.

42. U.S. Pat. No. 6,197,554 issued to Shi-Lung Lin et.al.

43. U.S. Pat. No. 6,130,040 issued to Shi-Lung Lin et.al.

44. U.S. Pat. No. 5,795,715 issued to Livache et.al.

45. Patent Cooperation Treaty Publication No. WO 00/75356 issued to Lin et.al.

46. U.S. Pat. No. 4,289,850 issued to Robinson.

47. U.S. Pat. No. 6,159,714 issued to Lau.

48. U.S. Pat. Nos. 4,945,082, 4,950652, 5,091,374 and 5,906,980 issued to Carter.

Gene silencing or inhibiting the expression of a gene holds great therapeutic and diagnostic promise. An example of this approach is antisense technology which can be used to inhibit gene expression in vitro and in vivo. However, many problems remain with development of effective antisense technology. For example, single-stranded DNA antisense oligonucleotides exhibit only short term effectiveness and are usually toxic at the doses required for biological effectiveness. Similarly, the use of single-stranded antisense RNAs has also proved ineffective due to its fast degradation and structural instability.

Other approaches to quelling specific gene activities are posttranscriptional gene silencing (PTGS) and RNA interference (RNAi) phenomena, which have been found capable of suppressing gene activities in a variety of in-vivo systems, including plants (Grant, S. R. (1999) Cell 96, 303-306), Drosophila melanogaster (Kennerdell, J. R. and Carthew, R. M. (1998) Cell 95, 1017-1026, Misquitta, L. and Paterson, B. M. (1999) Proc. Natl. Acad. Sci. USA 96, 1451-1456, and Pal-Bhadra, M., Bhadra, U., and Birchler, J. A. (1999) Cell 99, 35-46), Caenorhabditis elegans (Tabara, H., Sarkissian, M., Kelly, W. G., Fleenor, J., Grishok, A., and Timmons, L. (1999) Cell 99, 123-132, Ketting, R. F., Haverkamp, T. H., van Luenen, H. G., and Plasterk, R. H. (1999) Cell 99, 133-141, Fire, A., Xu, S., Montgomery, M. K., Kostas, S. A., Driver, S. E., and Mello, C. C. (1998) Nature 391, 806-811 and Grishok, A., Tabara, H., and Mello, C. C. (2000) Science 287, 2494-2497), zebrafish (Wargelius, A., Ellingsen, S., and Fjose, A. (1999) Biochem. Biophys. Res. Commun. 263, 156-161) and mouse (Wianny, F. and Zernicka-Goetz, M. (2000) Nature Cell Biol. 2, 70-75). In general, the transfection of a plasmid-like DNA structure (transgene) into cells induces PTGS phenomena, while that of a double-stranded RNA (dsRNA) causes an RNAi effect.

These phenomena appear to evoke an intracellular sequence-specific RNA degradation process, affecting all highly homologous transcripts, called cosuppression. It has been proposed that such cosuppression results from the generation of small RNA products (21˜25 nucleotide bases) by an RNA-directed RNA polymerase (RdRp) (Grant supra) and/or a ribonuclease (RNase) (Ketting et al. supra, Bosher, J. M. and Labouesse, M. (2000) Nature Cell Biology 2, 31-36, Zamore, P. D., Tuschl, T., Sharp, P. A. and Bartel, D. P. (2000) Cell 101, 25-33, and Elbashir et.al. (2001) Nature 411, 494-498) activity on an aberrant RNA template, derived from the transfecting nucleic acids or viral infection. Although an RdRp-independent endoribonucleolysis model has been proposed for the RNAi effect in Drosophila (Zamore, et al. supra), the RdRp homologues were widely found in Arabidopsi thalianas as Sde-1/Sgs-2 (Yang, D., Lu, H., and Erickson, J. W. (2000) Current Biology 10, 1191-1200), Neurospora crassa as Qde-1 (Cogoni, C. and Macino, G. (1999) Nature 399, 166-169) and Caenorhabditis elegans as Ego-1 (Smardon, A., Spoerke, J. M., Stacey, S. C., Klein, M. E., Mackin, N., and Maine, E. M. (2000) Curr. Biol. 10, 169-171). Thus, RdRp homologues appear to be a prerequisite for maintaining a long-term/inheritable PTGS/RNAi effect (Bosher, et al. supra).

Although PTGS/RNAi phenomena appear to offer a potential avenue for inhibiting gene expression, they have not been demonstrated to work well in higher vertebrates and, therefore, their widespread use in higher vertebrates is still questionable. All currently found RNAi effects are based on the use of double-stranded RNA (dsRNA), which have shown to cause interferon-induced non-specific RNA degradation (Stark et.al. (1998) Annu. Rev. Biochem. 67, 227-264, and Elbashir et.al. (2001) Nature 411, 494-498; U.S. Pat. No. 4,289,850 to Robinson and U.S. Pat. No. 6,159,714 to Lau). Such interferon-induced cellular response usually reduces the specific gene silencing effects of RNAi phenomena and may cause cytotoxic killing effects to the transfected cells. In mammalian cells, it has been noted that dsRNA-mediated RNAi phenomena are repressed by the interferon-induced RNA degradation when the dsRNA size is larger than 30 base-pairs or its concentrations are more than 10 nM (Elbashir supra). For therapeutic use such as prior arts U.S. Pat. Nos. 4,945,082, 4,950,652, 5,091,374 and 5,906,980 to Carter, the above limitations are critical to the determination of safe dosage for drug applications. It is impossible to deliver such small size and amount of dsRNAs in vivo due to the high RNase activities of our bodies. Consequently, there remains a need for an effective and sustained method and composition for inhibiting gene function in vivo in higher vertebrates.

To increase the efficiency and stability of gene silencing effects through RNAi phenomena, messenger RNA (mRNA)-complementary DNA (cDNA) hybrids have been proposed to be one of better candidates for such purpose than dsRNAs (Grant supra, Lin et.al. (1999) Nucleic Acid Res. 27, 4585-4589 and Alexeev et.al. (2000) Nat. Biotechnol. 18, 43-47). Although the mRNA-cDNA oligonucleotide has been shown to correct mutated gene expressions in mice skin (Alexeev et.al., Nat. Biotechnol. 18, 43-47 (2000)) and to silence oncogenes in human prostatic cancer cells (Lin et.al. (2001) Biochem. Biophys. Res. Commun. 281, 639-644), the mechanisms of these mRNA-cDNA oligonucleotides are actually based on recombinatory degradation which is not related to the RNAi phenomena (Lin et.al. (2001) Current Cancer Drug Targets in press). In summary, it is desirable to have a fast, stable and effective method for stimulating RNAi-related gene silencing effects, of which the results may be applied to screen special gene functions, to manipulate gene expressions in vitro, and even to design a therapy for genetic diseases in vivo.

### SUMMARY OF THE INVENTION

The present invention provides a novel composition and method for inhibiting gene function in higher eukaryotes in vivo. Without being bound by any particular theory, this method potentially is based on an RNAi-dependent gene silencing phenomenon, which is hereafter termed cDNA-aRNA interference. In accordance with the present invention, cDNA-aRNA hybrids are used for inhibiting gene function. The cDNA-aRNA hybrids of the present invention can be used to target a gene selected from the group consisting of functional genes, pathogenic nucleic acids, viral genes/genomes, bacterial genes, mutated genes, oncogenes and so on.

In specific embodiments, the present invention provides a method for gene silencing, comprising the steps of: a) providing: i) a substrate expressing a targeted gene, and ii) a composition comprising a cDNA-aRNA hybrid capable of silencing the expression of the targeted gene in the substrate; b) treating the substrate with the composition under conditions such that gene expression in the substrate is inhibited. The substrate can express the targeted gene in vitro or in vivo.

In one embodiment, the cDNA-aRNA hybrid targets a gene selected from the group consisting of functional genes, pathogenic nucleic acids, viral genes/genomes, bacterial genes, mutated genes, oncogenes. In another embodiment, the cDNA-aRNA hybrid inhibits b-catenin oncogene expression in human breast cancerous cells. In yet another embodiment, the cDNA-aRNA hybrid inhibits HIV-1 viral genome expression.

The present invention relating to cDNA-aRNA gene knockout technology can be used as a powerful new strategy in the field of gene-based therapy. The strength of this novel strategy is in its low dose, stability, and potential long-term effects. Applications of the present invention include, without limitation, the suppression of cancer related genes, the prevention and treatment of microbe related genes, the study of candidate molecular pathways with systematic knock out of involved molecules, and the high throughput screening of gene functions based on microarray analysis, etc. The present invention can also be used as a tool for studying gene function in physiological conditions.

The invention also provides compositions and methods for preparing cDNA-aRNA hybrids. Specifically, the present invention provides methods for generating cDNA-aRNA hybrids, comprising the steps of: a) providing: i) a solution comprising a nucleic acid template, ii) one or more primers sufficiently complementary to the sense conformation of the nucleic acid template, and iii) one or more promoter-linked primers sufficiently complementary to the antisense conformation of the nucleic acid template, and having an RNA promoter; b) treating the nucleic acid template with one or more primers under conditions such that a first cDNA strand is synthesized; c) treating the first cDNA strand with one or more promoter-linked primers under conditions such that a promoter-linked double-stranded nucleic acid is synthesized; d) treating the promoter-linked double-stranded nucleic acid under conditions such that essentially aRNA fragments are synthesized; and e) treating aRNA fragments with one or more primers under conditions such that a cDNA-aRNA hybrids are synthesized. The methods of the present invention can comprise the step of repeating steps b) through e) for a sufficient number of cycles to obtain a desired amount of amplified hybrid product.

The treating step in step b) can comprise heating the solution at a temperature above 90° C. to provide denatured nucleic acids. The treating step in step c) can comprise treating the first cDNA strand with one or more promoter-linked primers at a temperature ranging from about 37° C. to about 70° C., depending on the annealing sequence region used. The treating step in step c) can also comprise treating the cDNA strand with one or more promoter-linked primers in the presence of a polymerase.

In one embodiment, the polymerase is selected from the group consisting of DNA-dependent DNA polymerases, RNA-dependent DNA polymerases, RNA polymerases, Taq-like DNA polymerase, Tth-like DNA polymerase, C. therm. polymerase, viral replicases, and combinations thereof. The viral replicases can be selected from the group consisting of avian myeloblastosis reverse transcriptase and Moloney murine leukemia virus reverse transcriptase. In particular, the AMV reverse transcriptase does not have RNase H activity.

The treating step in step d) can comprise treating the promoter-linked double-stranded nucleic acid with an enzyme having transcriptase activity at about 37° C. The enzyme having transcriptase activity can be selected from the group consisting of RNA polymerases and viral replicases. The RNA polymerases can be selected from the group consisting of T3 RNA polymerase, T7 RNA polymerase, SP6 RNA polymerase, and M13 RNA polymerase.

The primers are complementary to the 3′-ends of the antisense conformation of the nucleic acid template. In one embodiment, one or more primers comprise a sequence-specific primer homologous to the targeted gene transcript.

The promoter-linked primers are complementary to the 3′-ends of the sense conformation of the nucleic acid template. In one embodiment, one or more promoter-linked primers comprise a sequence-specific primer complementary to the targeted gene transcript, such as T7 promoter-linked poly(dT) primers. The promoter-linked double-stranded nucleic acid template can be selected from the group consisting of linear and circular promoter-containing double-stranded DNAs or promoter-linked single-stranded DNAs.

In one embodiment, the treating step in step e) comprises treating aRNA fragments with one or more primers at a temperature ranging from about 37° C. to about 70° C., depending on the annealing sequence region used.

The methods of the present invention can further comprise the step of incorporating one or more nucleotide analogs into the cDNA portion of the cDNA-aRNA hybrid to facilitate the onset of RNAi-related effects. The nucleotide analog can be selected from the group consisting of inosine (I), xanthine(X), hypoxanthine (HX) and their derivative analogs. Alternatively, the nucleotide analog can be generated by adding deaminase or acidic chemicals to the cDNA portion of the cDNA-aRNA hybrid, resulting in derivatives selected from the group consisting of inosine (I) and its derivative analogs (See e.g., U.S. Pat. No. 6,130,040, incorporated herein in its single-stranding reaction by reference). In another embodiment, the methods of the present invention further comprise the step of contacting cDNA-aRNA hybrids with a reagent for gene silencing transfections. The reagent can be selected from the group consisting of electroporesis media, chemical transduction reagents and liposomal transfection reagents.

### BRIEF DESCRIPTION OF THE DRAWINGS

Referring particularly to the drawings for the purpose of illustration only and not limitation, there is illustrated:

FIG. 1 is an illustration of the preferred embodiment of the subject invention.

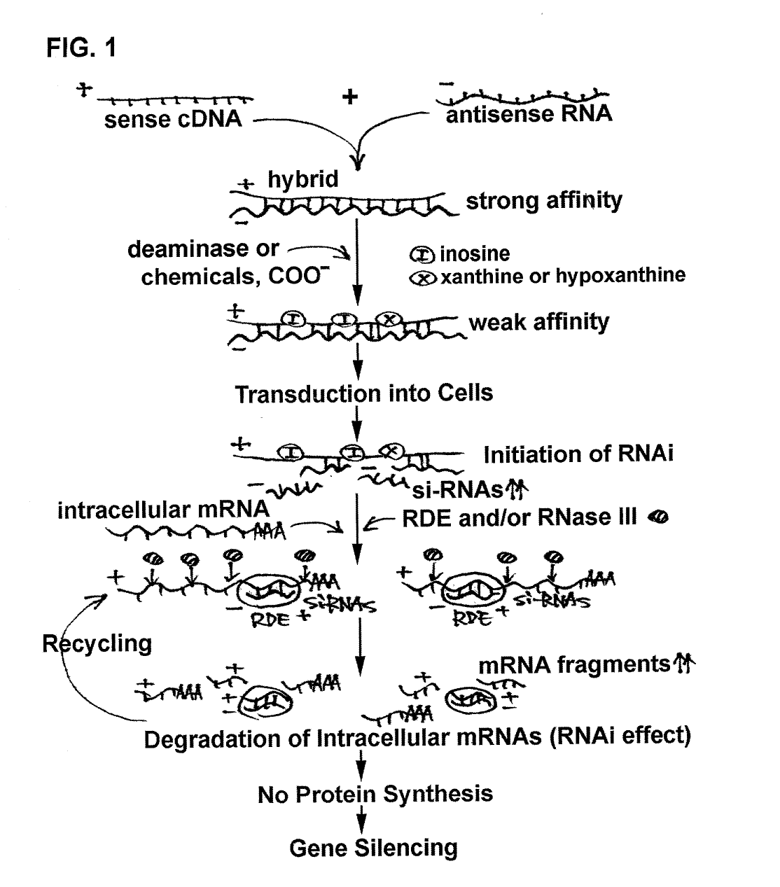

FIGS. 2a and 2b are the in-cell results of example 2 of the subject invention.

FIGS. 3a and 3b are the ex-vivo results of example 3 of the subject invention.

FIG. 4 is an illustration of the preferred embodiment of cDNA-aRNA generation of the subject invention.

FIG. 5 is the in-vivo result of example 4 of the subject invention.

### DEFINITIONS

To facilitate understanding of the invention, a number of terms are defined below.

As used herein, the terms “complementary” or “complementarity” are used in reference to polynucleotides (i.e., a sequence of nucleotides) related by the base-pairing rules. For example, the sequence “A-G-T” is complementary to the sequence “T-C-A.” Complementarity may be “partial,” in which only some of the nucleic acid bases are matched according to the base pairing rules. Alternatively, complementarity may be “complete” or “total” between the nucleic acids. The degree of complementarity between nucleic acid strands has significant effects on the efficiency and strength of hybridization between nucleic acid strands. This is of particular importance in amplification reactions, as well as in detection methods which depend upon binding between nucleic acids.

As used herein, the term “template” refers to a nucleic acid molecule being copied by a nucleic acid polymerase. A template can be single-stranded, double-stranded or partially double-stranded, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

As used herein, the term “nucleic acid template” refers to a double-stranded DNA/RNA, a single-stranded DNA, an mRNA/aRNA or an RNA-DNA hybrid.

As used herein, the term “primer” refers to an oligonucleotide complementary to a template. The primer complexes with the template to give a primer/template complex for initiation of synthesis by a DNA polymerase. The primer/template complex is extended by the addition of covalently bonded bases linked at its 3′ end, which are complementary to the template in DNA synthesis. The result is a primer extension product. Virtually all known DNA polymerases (including reverse transcriptases) require complexing of an oligonucleotide to a single-stranded template (“priming”) to initiate DNA synthesis.

As used herein, the term “promoter-linked primer” refers to an RNA-polymerase-promoter sense sequence coupled with a gene-specific complementary sequence in its 3′-end for annealing to the antisense conformation of a nucleic acid template.

As used herein, the term “DNA-dependent DNA polymerase” refers to an enzyme that synthesizes a complementary DNA copy from a DNA template. Examples are DNA polymerase I from E. coli and bacteriophage T7 DNA polymerase. Under suitable conditions a DNA-dependent DNA polymerase may synthesize a complementary DNA copy from an RNA template.

As used herein, the terms “DNA-dependent RNA polymerase” and “transcriptase” refer to enzymes that synthesize multiple RNA copies from a double-stranded or partially-double stranded DNA molecule having a promoter sequence. Examples of transcriptases include, but are not limited to, DNA-dependent RNA polymerase from E. coli and bacteriophages T7, T3, and SP6.

As used herein, the terms “RNA-dependent DNA polymerase” and “reverse transcriptase” refer to enzymes that synthesize a complementary DNA copy from an RNA template. All known reverse transcriptases also have the ability to make a complementary DNA copy from a DNA template. Thus, reverse transcriptases are both RNA-dependent and DNA-dependent DNA polymerases. As used herein, the term “RNase H” refers to an enzyme that degrades the RNA portion of an RNA/DNA duplex. RNase H's may be endonucleases or exonucleases. Most reverse transcriptase enzymes normally contain an RNase H activity in addition to their polymerase activity. However, other sources of the RNase H are available without an associated polymerase activity. The degradation may result in separation of RNA from a RNA/DNA complex. Alternatively, the RNase H may simply cut the RNA at various locations such that portions of the RNA melt off or permit enzymes to unwind portions of the RNA.

As used herein, the terms “hybridize” and “hybridization” refer to the formation of complexes between nucleotide sequences which are sufficiently complementary to form complexes via Watson-Crick base pairing. Where a primer (or splice template) “hybridizes” with target (template), such complexes (or hybrids) are sufficiently stable to serve the priming function required by the DNA polymerase to initiate DNA synthesis.

As used herein, the term “sense conformation” refers to a nucleic acid sequence in the same sequence order and composition as its homolog mRNA. The sense conformation is indicated as a “+” symbol.

As used herein, the term “antisense conformation” refers to a nucleic acid sequence complementary to its respective mRNA homologue. The antisense RNA (aRNA) refers to a ribonucleotide sequence complementary to an mRNA sequence in an A-U and C-G composition, and also in the reverse orientation of the mRNA. The antisense conformation is indicated as a “−” symbol.

As used herein, the term “gene” refers to a nucleic acid (e.g., DNA) sequence that comprises coding sequences necessary for the production of a polypeptide or precursor. The polypeptide can be encoded by a fall length coding sequence or by any portion of the coding sequence so long as the desired activity or functional properties (e.g., enzymatic activity, ligand binding, signal transduction, etc.) of the full-length or fragment are retained. The term “gene” encompasses both cDNA and genomic forms of a gene. A genomic form or clone of a gene contains the coding region interrupted with non-coding sequences termed “intervening regions” or “intervening sequences.”

As used herein, the term “gene silencing” refers to a phenomenon whereby a function of a gene is completely or partially inhibited. Throughout the specification, the terms “silencing,” “inhibition,” “quelling,” “knockout” and “suppression,” when used with reference to gene expression or function, are used interchangeably.

As used herein, the term “oligonucleotide” is defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

As used herein, the term “transfection” refers to the introduction of foreign DNA into eukaryotic cells. Transfection can be accomplished by a variety of means known to the art, including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

A primer is selected to be “substantially” or “sufficiently” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

As used herein, the term “amplification” refers to nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that will process only specific sequences of nucleic acid in a heterogeneous mixture of nucleic acid. For example, in the case of Qb replicase, MDV-1 RNA is the specific template for the replicase (Kacian et al. (1972) Proc. Natl. Acad. Sci. USA 69, 3038). Other nucleic acid will not be replicated by this amplification enzyme. Similarly, in the case of T7 RNA polymerase, this amplification enzyme has a stringent specificity for its own promoters (Chamberlin et al. (1970) Nature 228, 227). Taq and Pfu polymerases, by virtue of their ability to function at high temperature display high specificity for the sequences bounded, and thus defined by the primers.

As used herein, the terms “amplifiable nucleic acid” and “amplified products” refer to nucleic acids which may be amplified by any amplification method.

As used herein, the term “probe” refers to an oligonucleotide (i.e., a sequence of nucleotides) which is capable of hybridizing to another oligonucleotide of interest, whether occurring naturally as in a purified restriction digest or produced synthetically, recombinantly or by enzymatic amplification. A probe may be single-stranded or double-stranded. Probes are useful in the detection, identification and isolation of particular gene sequences.

As used herein, the term “enzymatic amplification” (such as PCR, NASBA and RNA-PCR) refers to a method for increasing the concentration of a segment in a target sequence from a mixture of genomic DNAs without cloning or purification (U.S. Pat. Nos. 4,683,195; 4,683,202; 4,965,188 (PCR); 5,888,779 (NASBA); 6,197,554 (RNA-PCR) and WO 00/75356, hereby incorporated by reference). This process for amplifying the target sequence consists of introducing a large excess of two oligonucleotide primers to the DNA mixture containing the desired target sequence, followed by a precise sequence of thermal cycling in the presence of DNA and/or RNA polymerase(s). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. Because the desired amplified segments of the target sequence become the predominant sequences (in terms of concentration) in the mixture, they are said to be amplified.

With enzymatic amplification, it is possible to amplify a single copy of a specific target sequence in genomic DNA to a level detectable by several different methodologies (e.g., incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR and RNA-PCR process itself are, themselves, efficient templates for subsequent PCR and RNA-PCR amplifications.

As used herein, the term “portion” when in reference to a protein or nucleic acid sequence refers to fragments of that protein or nucleic acid sequence. Fragments of a protein can range in size from four amino acid residues to the entire amino acid sequence minus one amino acid.

The term “nucleotide analog” as used herein refers to modified or non-naturally occurring nucleotides such as 7-deaza purines (i.e., 7-deaza-dATP and 7-deaza-dGTP). Nucleotide analogs include base analogs and comprise modified forms of deoxyribonucleotides as well as ribonucleotides.

The term “Northern blot,” as used herein refers to the analysis of RNA by electrophoresis of RNA on agarose gels to fractionate the RNA according to size, followed by transfer of the RNA from the gel to a solid support such as nitrocellulose or a nylon membrane. The immobilized RNA is then probed with a labeled probe to detect RNA species complementary to the probe used. Northern blots are a standard tool of molecular biologists (Sambrook et al., (1989) Molecular Cloning, 2^{nd} Ed., Cold Spring Harbor Laboratory Press, pp 7.39-7.52).

As used herein, the term “Southern blot” refers to the analysis of DNA on agarose or acrylamide gels to fractionate the DNA according to size, followed by transfer of the DNA from the gel to a solid support such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., supra).

The term “virus” refers to obligate, ultramicroscopic, intracellular parasites incapable of autonomous replication (i.e., replication requires the use of the host cell's machinery).

As used herein, the terms “Taq-like polymerase” and “Taq polymerase” refer to Taq DNA polymerase and derivatives. Taq DNA is widely used in molecular biology techniques including recombinant DNA methods. For example, various forms of Taq have been used in a combination method which utilizes PCR and reverse transcription (See e.g., U.S. Pat. No. 5,322,770, incorporated herein in its entirety by reference). DNA sequencing methods which utilize Taq DNA polymerase have also been described. (See e.g., U.S. Pat. No. 5,075,216, incorporated herein in its entirety by reference).

As used herein, the terms “Tth-like polymerase” and “Tth polymerase” refer to polymerase isolated from Thermus thermophilus. Tth polymerase is a thermostable polymerase that can function as both reverse transcriptase and DNA polymerase (Myers and Gelfand, (1991) Biochemistry 30, 7662-7666). It is not intended that the methods of the present invention be limited to the use of Taq-like or Tth-like polymerases. Other thermostable DNA polymerases which have 5′ to 3′ exonuclease activity (e.g., Tma, Tsps17, TZ05, Tth and Taf) can also be used to practice the compositions and methods of the present invention.

### DESCRIPTION OF THE PREFERRED EMBODIMENT

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

The present invention also relates to compositions and methods for generating cDNA-aRNA hybrids (Lin (1999) supra) and compositions and methods using the same for gene silencing, named “D-aRNAi”. The mechanism of D-aRNAi relies on the exogenous transfection of aberrant RNAs to stimulate intracellular defense system (such as posttranscriptional gene silencing (PTGS) effects) against such transfection (Grant (1999) supra). In brief, the intracellular defense system directs an RNA-dependent RNA polymerase (RdRp) or RNA-directed endoribonuclease (RDE) to generate many short RNA fragments (si-RNAs) from the aberrant RNA template (herein the cDNA-aRNA hybrids in this preferred embodiment). The si-RNA can be further targeted by the RDE (or RNase III) for the fast degradation of its homologous gene transcripts (Scott W. Knight and Brenda L. Bass (2001) Science 293, 2269-2271). However, because the RNA construct itself is highly susceptible to fast degradation and the RdRp/RDE is more sensitive to double-stranded templates, current scientists prefer to use double-stranded RNA (dsRNA; Fire, supra) or mRNA-cDNA hybrid (D-RNAi; Lin (2001) supra) as an aberrant template for better transfection results.

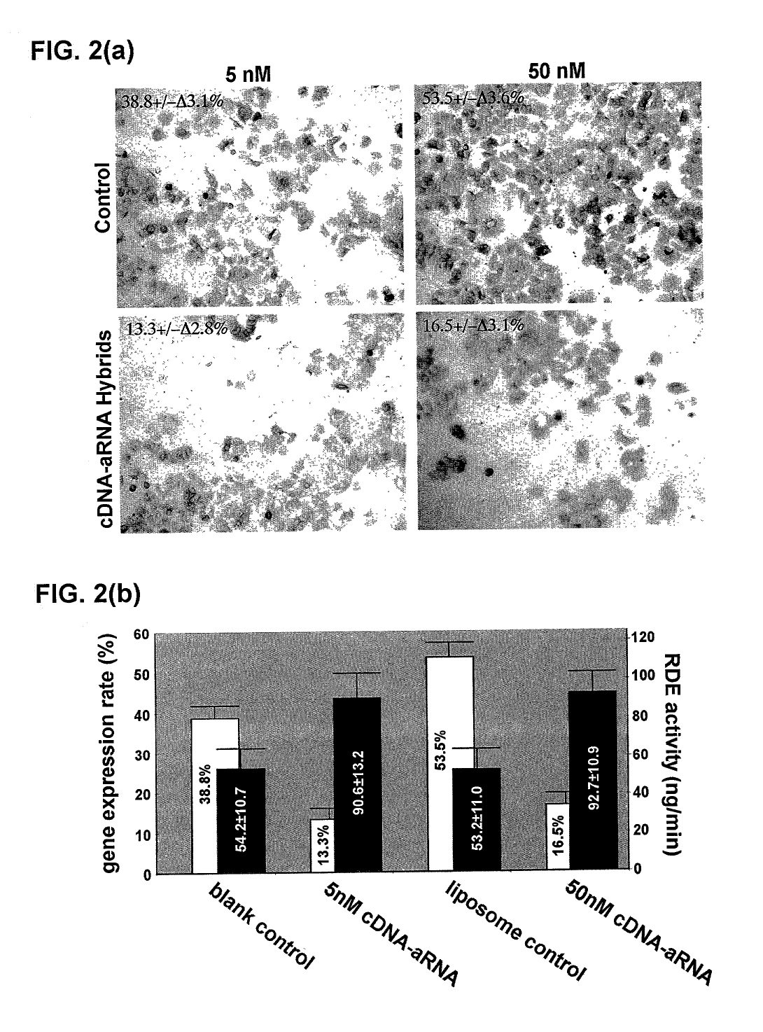

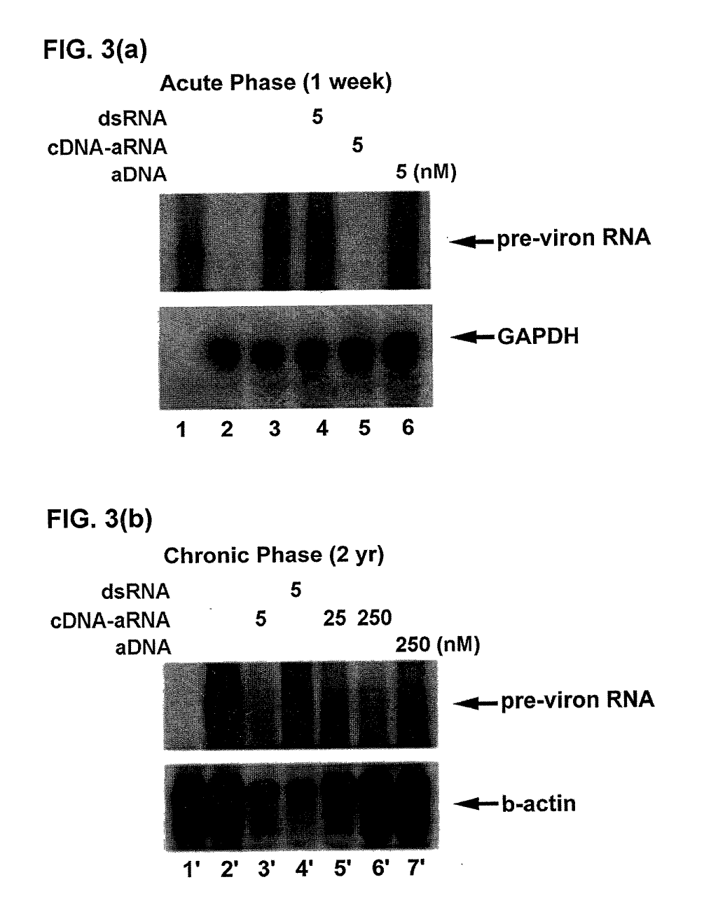

Because previous dsRNA transfection experiments showed significant interferon-induced cytotoxicity (Stark and Elbashir supra), we have successfully overcome this problem and induced specific gene silencing effects in human cell models using a more stable cDNA-aRNA hybrid construct. The advantages of cDNA-aRNA hybrid over dsRNA transfection are listed as follows: 1) the cDNA portion of a cDNA-aRNA hybrid can be modified to increase the efficacy of RNAi phenomenon induction; 2) the RNA portion of a cDNA-aRNA hybrid is well protected by the cDNA portion of the same for more stable transfection (Lin (2001) supra); 3) the RNAi-associated RNA-directed endoribonuclease has experimentally shown to possess higher activity to the aRNA portion of a cDNA-aRNA hybrid (see FIG. 2(b)); 4) the cDNA-aRNA hybrid construct has been tested to suppress the interferon-induced cytotoxicity which is usually caused by dsRNA (see FIG. 3(b) and Example 3); and 5) the size of a cDNA-aRNA hybrid can be larger than 30 base pairs for more effective transfection and specific gene targeting.

To facilitate the onset of RNAi-related effects in cells, the cDNA portion of a cDNA-aRNA hybrid can be modified to increase the efficiency of release of the aRNA portion to a RNAi-associated RNA-directed endoribonuclease (RDE). Such modification can be accomplished either by the incorporation of weak binding nucleotide analogs during the synthesis of the cDNA portion or the deamination of cDNA sequence nucleotides after its synthesis. For the incorporation method, the nucleotide analogs are integrated into the cDNA sequence using a oligonucleotide synthesizer machine (e.g. SEQ ID.3) or an enzymatic reaction, such as reverse transcription (RT), polymerase chain reaction (PCR), nucleic acid sequence based amplification (NASBA) and RNA-polymerase cycling reaction (RNA-PCR) (e.g. Example 3). The nucleotide analog can be selected from the group consisting of inosine (I), xanthine(X), hypoxanthine (HX) and their derivative analogs. Alternatively, the nucleotide analog can be generated by adding deaminase or acidic chemicals (e.g. acetic acid) to the cDNA sequence, resulting in derivative analog(s) selected from the group consisting of inosine (I) and its derivatives (See e.g., U.S. Pat. No. 6,130,040).

The cDNA-aRNA hybrids of the invention are preferably prepared using an improvement of the so-called RNA-PCR described in U.S. Pat. No. 6,197,554.

RNA-Polymerase Chain Reaction (RNA-PCR)

A thermocycling amplification-like reaction performed on RNAs, or called RNA-polymerase cycling reaction, to provide a highly efficient amplification (>250-fold/cycle) of the RNA sequences existing in the prior art. (Lin, (1999) Nucl. Acids. Res. 27, 4585-4589; U.S. Pat. No. 6,197,554 to Lin et al., incorporated herein by reference in their entirety). The elevated thermocycling temperature prevents rapid degradation of short-lived RNAs and further reduces the secondary structure of RNAs to increase the accessibility of enzyme interactions and the production of more complete full-length RNAs. The procedure uses thermostable enzymes, including Tth-like polymerases with reverse transcriptase activity. The use of proofreading RNA polymerases for amplification not only provides higher fidelity but also eliminates preferential amplification of abundant RNA species. Additionally, rapid and simple cell fixation and permeabilization steps inhibit any alterations in gene expression during specimen handling or genomic contamination. (See, Embleton et al., (1992) Nucl. Acids Res. 20, 3831-3837).

The procedure was implemented using a poly(dT)₂₄ primer to generate the first-strand cDNAs. Another oligo(dC)-promoter primer is used to generate the second-strand cDNAs. Both strands together form the promoter-linked double-stranded cDNAs from the original mRNAs. The oligo(dC)-promoter primer is an equal mixture of oligo(dC)₁₀N sequences (N=dG, dA or dT) coupled to an RNA promoter for in vitro transcription along the double-stranded cDNA templates. These amplified mRNAs not only share the same properties but also have the full integrity of their original mRNAs, depending on the quality of the first promoter-linked double-stranded cDNAs.

RNAs generated with this procedure are well amplified and well represented. According to the high efficiency of transcriptional amplification (up to 2000-fold/cycle), three rounds of RNA-PCR are theoretically equivalent to 33 cycles of PCR amplification (2-fold/cycle). Theoretically, a single copy of mRNA can be multiplied more than 1 billion-fold. Thirty mg of amplified mRNAs have been acquired in one 50 ml reaction after three rounds of RNA-PCR amplification from 20 cells. This represents a 15 million-fold increase based upon a comparison between the amount of synthesized mRNAs and that of theoretically presumed mRNAs within a cell (0.1 pg).

Like other promoter-driven nucleic acid sequence amplification methods, RNA-PCR is also useful for cloning a specific nucleic acid sequence. (See e.g., Lin et al., Biochem. Biophys. Res. Commun., 256: 187-192 (1999)); Kwoh et al., Proc. Natl. Acad. Sci. USA 86: 1173-1177 (1989)). When performed with a specific primer complementary to the 3′-margin of the desired RNA sequence, RNA or cDNA fragments of an appropriate size can be generated by RNA-PCR for further genetic analysis. The design of these sequence-specific primers is based on the same principles used for PCR. Since reverse transcriptase extension of the specific primers forms RNA-cDNA hybrids and in vitro transcription through the promoter primers forms mRNAs, the desired single-stranded nucleotide probes by adding either DNase or RNase to digest the unwanted half of the amplified products.

Further, RNA-PCR is now routinely used to generate high purity nucleotide probes because it alternates between synthesizing RNAs and cDNAs, depending on the stopping point chosen during the amplification cycle. The labeling of cDNAs can be easily accomplished by incorporating labeled nucleotide analogs during reverse transcription, whereas the labeling of RNAs is completed during transcription. Typically, these probes can be useful in a variety of applications, such as hybridization blotting, gene knock-out transfection, in situ detection and genetic cloning (Lin (1999) supra).

Methods For Generating cDNA-aRNA Hybrids for Gene Silencing

The present invention provides a simple, fast, and inexpensive method for amplifying specific cDNA-aRNA hybrids for gene silencing transfection. The cDNA-aRNA hybrids can be used for screening special gene functions, for manipulating gene expressions in vitro, and for designing a therapy for genetic diseases in vivo.

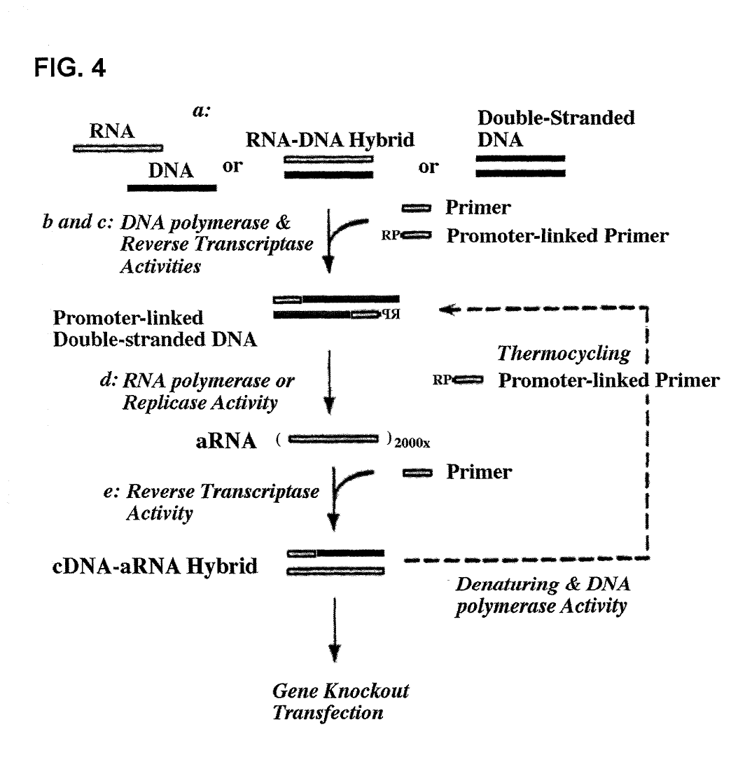

The present invention is also directed to an improved RNA-polymerase cycling reaction method for generating cDNA-aRNA hybrid duplexes (FIG. 4) for gene interference effects in living cells. By modifying our RNA-PCR methods (Lin (1999) supra), we use a gene-specific primer and promoter-primer in a thermocycling procedure to amplify specific cDNA-aRNA sequences for gene knockout technologies. This thermocycling procedure preferably starts from reverse transcription of mRNAs with RNA promoter-containing primer(s) and Tth-like polymerases, following cDNA double-stranding reaction with the same Tth-like polymerases. The resulting promoter-linked double-stranded DNAs are served as transcriptional templates for amplifying aRNA amount up to 2000 fold/cycle by RNA polymerases. The thermocycling procedure can be repeated for more amplification of the cDNA-aRNA hybrids. Alternatively, we can use a promoter- and template-containing vector for aRNA preparation directly by RNA polymerases and reverse transcribe the amplified aRNAs for further applications, such as gene interference analysis, gene-specific sequence detection and potential gene therapy.

The amplification cycling procedure of the present invention presents several advantages over prior amplification methods. First, cDNA-aRNA probes from low-copy rare mRNA species can be prepared within three round of amplification cycling without mis-reading mistakes. Second, the cDNA-aRNA hybrid amplification is linear and does not result in preferential amplification of nonspecific gene sequences. Third, the RNA degradation is inhibited by thermostable enzymatic conditions with RNase inhibitors. And the last, the use of RNase H activity is restricted in this preferred embodiment to preserve the integrity of final cDNA-aRNA constructs. Unlike previous NASBA methods (Compton, Nature 350: 91-92 (1991)), this improved RNA-PCR procedure contains no RNase H activity which usually destroys the RNA structure of a RNA-DNA hybrid. Based on these advantages, we therefore can use the present invention to prepare high amount of pure and specific cDNA-aRNA hybrids for transducing biological effects of interest in vitro, ex vivo as well as in vivo.

The labeling of cDNA-aRNA hybrids is accomplished by incorporation of labeled nucleotides or analogs during the reverse transcription of aRNAs. The nucleotide sequences so generated are useful for tracking down the transfected cells in a large cell population. These labeled nucleotides are also capable of being probes in a variety of applications, such as Southern blots, dot hybridization, position cloning, nucleotide sequence detection, gene knockout transfection and so on. The incorporated nucleotide analogs also provide better protection of the cDNA-aRNA structures, resulting in more stability and effectiveness of the probe transfection. The nucleotide analog can be selected from the group consisting of biotin-labeled, digoxigenin-labeled, fluorescein-labeled, amino-methylcoumarin-labeled, tetramethyl-rhodamine-labed nucleotides and their derivatives.

In the preferred embodiments (FIG. 4) of the present invention, according to the high amplification efficiency of RNA polymerase (up to 2000 folds/cycle), the labor- and time-consuming factors in this cDNA-aRNA hybrid generation method can be reduced to the minimum. Also, such preparation of amplified cDNA-aRNA hybrids is much cheaper and more efficient than traditional cDNA cloning with an expression-competent plasmid vector and then reverse transcription of the expressed RNA products. Most importantly, this cDNA-aRNA hybrid amplification can be carried out in a microtube with only a few nucleic acid template (0.2 pg). Taken together, these special features make the improved content of RNA-PCR as simple, fast, and inexpensive as a kit for concisely isolating amplified cDNA-aRNA hybrid sequences for specific gene knockout assays.

Although certain preferred embodiments of the present invention have been described, the spirit and scope of the invention is by no means restricted to what is described above. For example, within the general framework of: a) one or more types of nucleic acid templates used; b) one or more specific primers for reverse transcription and polymerase extension reactions; c) one or more promoter-linked primers for transcription reactions; d) one or more enzymes for each step of reaction(s); e) one or more rounds of the cycling procedure for cDNA-aRNA hybrid amplification, there is a very large number of permutations and combinations possible, all of which are within the scope of the present invention.

Gene Silencing Using cDNA-mRNA Hybrids: In Vitro Breast Cancer Model

As noted earlier, posttranscriptional gene silencing (PTGS) and RNA interference (RNAi) have been found capable of quelling specific gene activities in a variety of in vivo systems.

According to the invention provided herein, ectopic transfection of a sequence-specific cDNA-aRNA hybrid (instead of a transgene dsDNA or dsRNA) is used to induce intracellular gene silencing in human cells. Although previous transgene/dsRNA transfection experiments showed that PTGS/RNAi effects are limited to plants and some simple animals, using the present invention, specific gene interference of β-catenin expression in human MCF-7 breast cancer cells using the cDNA-aRNA hybrid transfection has been successfully detected.

Normal human mammary granular cells do not express β-catenin protein, whereas neoplastic breast tissues from late-stage patients show a highly elevated level of this proliferation-stimulating oncoprotein. The malignancy and metastatic potentials of the breast cancer cells are also significantly increased after β-catenin expression. It is known in the art that over-expression of β-catenin protects malignant cancer cells from apoptosis and confers resistance to many anti-cancer drugs in vivo. To overcome such resistance, anti-sense gene therapy may provide a counteract control for the expression of β-catenin.

The potential utility of cDNA-aRNA transfection in preventing oncogene expression was therefore tested on β-catenin-expressing MCF-7 cells, expecting to reduce β-catenin protein amount and increase cancer cell susceptibility to apoptotic stimuli. Following our previous findings, MCF-7 cells were treated with different dosages of anti-b-catenin cDNA-aRNA hybrids (5 nM at an optimally effective concentration and 50 nM at a ten-fold high concentration). FIG. 2(a) shows the immunostaining results of expressed β-catenin protein in red ACE substrate color. At 5 nM concentration of the cDNA-aRNA transfection (n=4), the expression rate was decreased from 38.8±3.1% (control) to 13.3±2.8% (transfected) cell population, indicating a 65.8% reduction. At 50 nM concentration (n=5), the expression rate was decreased from 53.5±3.6% (control) to only 16.5±3.1% (transfected) cell population, indicating a 69.3% reduction.

The silencing of β-catenin expression also decrease the proliferation rate of cancer cells. At 5 nM concentration of the cDNA-aRNA transfection (n=4), the density of cell population was decreased from average 112 (control) to 43 cell/mm³ (transfected), indicating a 62.7% reduction. At 50 nM concentration (n=5), the density of cell population was decreased from average 155 (control) to only 37 cell/mm³ (transfected) cell population, indicating a 76.2% reduction. It is also noted that the cell morphology of all four sets is the same, without the debris of apoptotic bodies (interferon-caused cell death). Such findings suggest that the cDNA-aRNA transfection can successfully knock out average 67% of β-catenin oncogene expression and inhibit more than 62% cancer cell growth without the induction of cytotoxicity. Contrary to previous dsRNA reports, dsRNA transfection usually causes a very significant interferon-induced cytotoxicity at the concentrations more than 10 nM.

The increase of RNA-directed endoribonuclease (RDE) activity is also detected after cDNA-aRNA transfections. As noted earlier, the RDE is required for the onset of PTGS/RNAi phenomena in many in cell and in vivo systems. The activity of RDE is measured by adding 2 μl cell extracts into 2 μg of 1 kb dsRNA preparations for 10 min at 25° C. Since the dsRNA is labeled by [³³P]-CTP (>3000 Ci/mM, Amersham International), the degradation rate can be easily observed by 1% agarose gel electrophoresis, blot transferring and then film exposure. The bar chart of FIG. 2(b) shows the RDE activity in black bars and the gene silencing rate in white bars. At 5 nM concentration of the cDNA-aRNA transfection (n=4), the RDE activity was promoted from 54.2 (control) to 90.6 ng/min (transfected), indicating a 167% increase rate. At 50 nM concentration (n=5), the RDE activity was promoted from 53.2 (control) to 92.7 ng/min (transfected), indicating a 174% increase rate. This data suggests that the cDNA-aRNA transfection induce a gene-specific silencing effect through the PTGS/RNAi phenomena.

There are three major effects of PTGS, i.e., initiation, spreading and maintenance, all of which are also found in many inheritable RNAi phenomena. The initiation indicates that the onset of PTGS/RNAi takes a relatively long period of time (1˜3 days) to develop enough small RNA or short aRNA (si-RNA) for specific gene knockout. With traditional antisense transfection processes, it only takes several hours to reach the same gene silencing results but with much higher dosages and higher cytotoxicity. Also, unlike the short-term effectiveness of traditional antisense transfections, the PTGS/RNAi effects may spread from a transfected cell to neighboring cells and can be maintained for a very long time (weeks to lifetime) in a mother cell as well as its daughter cells (Grant (1999) supra). Based on these features, a more efficient and reliable gene therapy is expected.

Gene Silencing Using cDNA-aRNA Hybrids: Ex Vivo Model Targeting HIV-1 Genome in CD4⁺ Tc Lymphocyte Extracts

The foregoing establishes that the novel cDNA-aRNA hybrids of the present invention can be used in a novel strategy to knock out targeted gene expression in vitro. As discussed below, the novel cDNA-aRNA strategy of the invention is also effective in knocking out gene expression ex vivo.

As illustrated in the examples below, the methods and compositions of the invention are effective in knocking out exogenous viral gene expression ex vivo in a CD4⁺ Tc lymphocyte extract model. For molecules, HIV-1 genome from +1890 to +2230 bases was targeted because it has a critical role in viral replication activity, and for cells, CD4⁺ Tc lymphocyte was selected because it is a major favor for HIV-1 infection. The HIV-1 is known to be the infectious pathogen of AIDS diseases. To a world-wide estimation till year 2000, more than 36 million people are currently infected by HIV-1, and this number is increased by at least 2 million per year. About four million AIDS patients have deceased this year due to the lack of an effective and stable long-term treatment for eradicating the malignancy of this virus.

The high mutation rate of HIV genome gradually generates more and more unexpected resistance to traditional HAART cocktail therapy, exacerbating the prevalence of this disease. Such dramatic increase of new mutant viruses as well as their carriers will soon become a very heavy finance burden for all health care and related disease prevention programs. However, although the high mutation rate of HIV-1 genome enable it to escape the traditional chemotherapy, it is impossible for HIV to change the whole targeted sequence which is more than 300 bases homologous to our cDNA-aRNA probe. Because the cosuppression effect of RNAi phenomenon to all homologous transcripts, the HIV genes is impossible to evade the silencing effects of cDNA-aRNA transfection by its mutations. It is very promising that the cDNA-aRNA transfection could become a powerful antiviral drug or vaccine for the prevention, or therapy, of viral infections.

FIG. 3(a) shows the gene silencing effect of anti-HIV1 cDNA-aRNA transfections (n=3 for each set) in acute phase AIDS patient Tc lymphocyte extracts, while FIG. 3(b) shows the same effect in chronic phase AIDS patient Tc lymphocyte extracts. The lane 1 of FIG. 3(a) is pure HIV-1 genome to indicate the size location on an electrophoresis gel. The lane 2 of FIG. 3(a) and lane 1′ of FIG. 3(b) are Tc lymphocyte RNA extract samples from normal non-infected persons as negative control. The lane 3 of FIG. 3(a) and lane 2′ of FIG. 3(b) are extract samples from HIV1-infected patients as positive control. In the acute phase (one-week infection), the treatment of 5 nM cDNA-aRNA transfection knocks out almost all viral gene expression, while those of 5 nM dsRNA and traditional antisense DNA transfection have very minor effects. In the chronic phase (two-year infection), the treatment of 5 nM cDNA-aRNA transfection knocks out 55.8% viral gene expression, while the transfections of 5 nM dsRNA and 250 nM traditional antisense DNA have no effects. When the cDNA-aRNA concentration is increased to 250 nM (FIG.(3b), lane 6′), the transfection knocks out 61.3% viral gene expression without the induction of cytotoxicity. The expression of cellular house-keeping genes, GAPDH and β-actin, is normal and shows no interferon-induced non-specific RNA degradation in most of lanes, except the dsRNA treatments. These findings have directed to an immediate therapy potential for AIDS in both acute and chronic infections.

As discussed above, the experimental results establish that cDNA-aRNA hybrids potentially inhibit β-catenin expression in the MCF-7 cancer cells and also prevent HIV-1 viral activity in the CD4⁺ Tc lymphocytes. Thus, the results show that using a cDNA-aRNA duplex provides a powerful new strategy for gene therapy. At the highest dosage used in the experiments here (FIGS. 2 and 3), the cDNA-aRNA transfection did not cause interferon-induced cytotoxicity as previous reports in dsRNA transfections. This even underscores the fact that the cDNA-aRNA comprising compositions of the instant invention are effective even at low dosages. The results also indicate that this invention is effective in knocking out the targeted gene expression over a relatively long period of time. Further, it was observed that non-targeted cells appear to be normal, which implies that the compositions herein possess no overt toxicity. Thus, the invention offers the advantages of low dosage, stability, long term effectiveness, and lack of overt toxicity.

Gene Silencing Using cDNA-aRNA Hybrids: In Vivo Model Interferencing Tyrosinase Gene Expression in Mouse Skin Hairs.

The foregoing establishes that the novel cDNA-aRNA hybrids of the present invention can be used in a novel strategy to knock out targeted gene expression in vitro as well as ex vivo. As discussed below, the novel cDNA-aRNA strategy of the invention is also effective in knocking out gene expression in vivo.

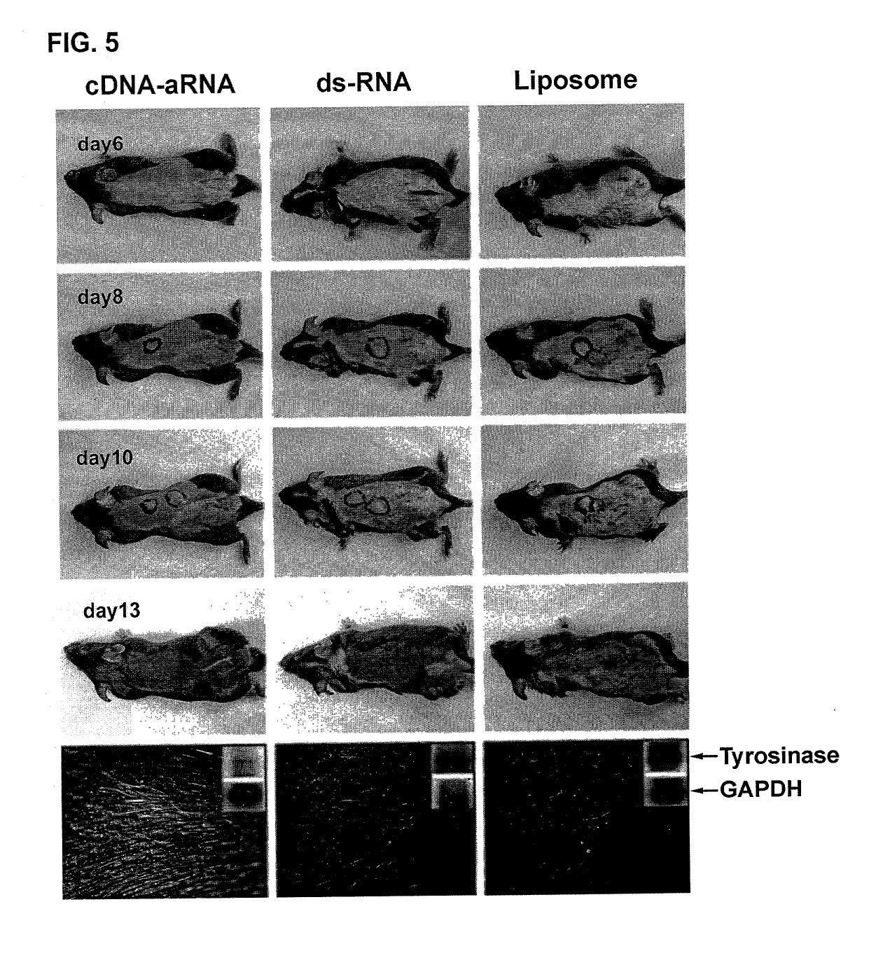

As illustrated in the examples below, the methods and compositions of the invention are effective in knocking out specific gene expression in vivo in a mouse skin hair model. As shown in FIG. 5, albino (white) skin hairs of melanin-knockout mice were created by four times of intra-cutaneous (i.c.) transduction of about 50 nM mismatched cDNA-aRNA per day against tyrosinase (tyr) gene transcripts. The expression of melanin(black pigment) in skins and hairs has been blocked due to a loss of its intermediate generation by the tyrosinase knockout. Contrarily, the control and double-stranded RNA (dsRNA) transfected mice presented normal skin color (black), indicating that the loss of melanin is specific to RNAi silencing effect induced by the cDNA-aRNA transfection. Moreover, Northern blotting showed a 76.1±5.3% reduction of tyr gene expression after the cDNA-aRNA transfection, while minor non-specific degradation of common gene transcripts (such as GAPDH) was detected in the dsRNA transfected skins.

As discussed here, the experimental results establish that cDNA-aRNA hybrids potentially inhibit tyrosinase gene expression in the transfected mice skins and therefore prevent the production of melanin (black pigment) in hairs. Thus, the results show that using a cDNA-aRNA duplex provides a powerful new strategy for gene therapy, especially to melanoma. At the same dosage (200 nM in total), the cDNA-aRNA transfection did not cause any cytotoxicity effect, while the dsRNA transfections induced detectable non-specific mRNA degradation as previous reports (Stark (1998) supra, and Elbashir (2001) supra). This even underscores the fact that the cDNA-aRNA comprising compositions of the instant invention are effective even under in vivo systems without the side-effects of dsRNA. The results also indicate that this invention is effective in knocking out the targeted gene expression over a relatively long period of time because the hair regrowth requires at least ten-day recovery. Further, it was observed that non-targeted skin hairs appear to be normal, which implies that the compositions herein possess high specificity and no overt toxicity. Thus, the invention offers the advantages of low in-vivo dosage, stability, long term effectiveness, and lack of overt toxicity.

### EXPERIMENTAL

The following examples serve to illustrate certain preferred embodiments and aspects of the present invention and are not to be construed as limiting the scope thereof.

In the experimental disclosure which follows, the following abbreviations apply: M (molar); mM (millimolar); mm (micromolar); mol (moles); pmol (picomolar); gm (grams); mg (milligrams); L (liters); ml (milliliters); ml (microliters); ° C. (degrees Centigrade); cDNA (copy or complimentary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); NaCl (sodium chloride); HEPES (N-2-hydroxyethylpiperazine-N-2-ethanesulfonic acid); HBS (HEPES buffered saline); SDS (sodium dodecylsulfate); Tris-HCl (tris-hydroxymethylaminomethane-hydrochloride); and ATCC (American Type Culture Collection, Rockville, Md.).

All routine techniques and DNA manipulations, such as gel electrophoresis, were performed according to standard procedures. (See Sambrook et al., supra). All enzymes and buffer treatments were applied following the manufacture's recommendations (Roche Biochemicals, Indianapolis, Ind.). For Northern blots, mRNAs were fractionated on 1% formaldehyde-agarose gels and transferred onto nylon membranes (Schleicher & Schuell, Keene, N.H.). Probes were labeled with the Prime-It II kit (Stratagene, La Jolla, Calif.) by random primer extension in the presence of [³²P]-dATP (>3000 Ci/mM, Amersham International, Arlington Heights, Ill.), and purified with Micro Bio-Spin chromatography columns (BIO-RAD, Hercules, Calif.). Hybridization was carried out in the mixture of 50% freshly deionized formamide (pH 7.0), 5×Denhardt's solution, 0.5% SDS, 4×SSPE and 250 mg/ml denatured salmon sperm DNAs (18 hr, 42° C.). Membranes were sequentially washed twice in 2×SSC, 0.1% SDS (15 min, 25° C.), and once each in 0.2×SSC, 0.1% SDS (15 min, 25° C.); and 0.2×SSC, 0.1% SDS (30 min, 65° C.) before autoradiography by films.

For cell fixation and permeabilization, MCF-7 cells, a breast cancer cell line, were grown in MEM medium supplemented with 10% fetal calf serum. A sample containing cells cultured in a 60 mm dish (70% full of cells) was trypsinized, collected and washed three times in 5 ml phosphate buffered saline (PBS, pH 7.2) at room temperature. After washing, the cells were suspended in 1 ml of ice-cold 10% formaldehyde solution in 0.15M NaCl. After one hour incubation on ice with occasional agitation, the cells were centrifuged at 13,000 rpm for 2 min, and washed three times in ice-cold PBS with vigorous pipetting. The collected cells were resuspended in 0.5% non-ionic detergents, such as (octylphenoxy)-polyethanol or polyoyethylenesorbitan (Sigma), and incubated for one hour with frequent agitation. The cells were washed three times in ice-cold PBS containing 0.1M glycine, then resuspended in 1 ml of the same buffer with vigorous pipetting in order to be evenly separated into small aliquots and stored at −70° C. for up to a month.

### EXAMPLE 1

### The Preparation of cDNA-aRNA Hybrids

Few fixed and permeabilized MCF-7 cells were applied to a reaction (20 μl) on ice, comprising 2 μl of 10×RT&T buffer (400 mM Tris-HCl, pH 8.3 at 25° C., 350 mM KCl, 80 mM MgCl₂, and 100 mM DTE), 1 μM β-catenin-antisense promoter-linked primer 5′-dAAACGACGGC CAGTGAATTG TAATACGACT CACTATAGGC GCTCTGAAGA CAGTCTGTCG TGATGG-3′ (SEQ ID.1), 1 μM β-catenin-sense primer 5′-dATGGCAACCC AAGCTGACTT GATC-3′ (SEQ ID.2), ribonucleotide triphosphates (4 mM each for ATP GTP, CTP and UTP), deoxyribonucleotide triphosphates (4 mM each for dATP dGTP, dCTP and dTTP), and RNase inhibitors (10U). After C. therm./Taq DNA polymerase mixture (4U) was added, the reaction was incubated at 52° C. for 3 min, 65° C. for 30 min, 94° C. for 3 min, 52° C. for 3 min and then 68° C. for 3 min. A transcription reaction was prepared by adding T7 RNA polymerase (200U) and C. therm. polymerase (6U) mixture into above reaction. After three-hour incubation at 37° C., the resulting antisense RNA transcripts were continuously reverse-transcribed into cDNA-aRNA hybrids at 52° C. for 3 min and then 65° C. for 30 min. The quality of amplified cDNA-aRNA products can be assessed on a 1% formaldehyde-agarose gel (Lin (1999) supra).

### EXAMPLE 2

### In-Cell Transfection and Gene Silencing Induction

Above β-catenin cDNA-aRNA hybrid probe (10 μg) was treated by deaminase (10U, New England BioLab) for 30 min at 37° C. in 0.5×RT&T buffer. The resulting product was purified by microcon-30 filter, dissolved in 75 μl of Hepes buffer (pH 7.4) and mixed with 50 μl of DOTAP liposome (1 mg/ml, Roche Biochemicals) on ice for 30 min before applied to 60 mm (2 ml) diameter culture dishes which contain 50% confluency of MCF-7 cancerous cells. The MCF-7 cells were grown in MEM medium with 10% bovine serum. After 72 hr incubation, the gene expression of β-catenin protein was shown by immuno-histochemical staining with 50 μg/ml anti-β-catenin antibodies (Santa Cruz BioLab) and found to be reduced more than 66˜70% in the cDNA-aRNA hybrid set while the blank and liposomal control sets have no significant gene silencing effects (FIG. 2(a)). The RNA-directed endoribonuclease (RDE) activity of the cDNA-aRNA hybrid transfection set was also detected to show a 167.2˜174.2% increase following the reduction of β-catenin expression (FIG. 2(b)). Such increase of RDE activity reflects a high RNAi effect induced by the cDNA-aRNA hybrid transfection. Because the over-expression of β-catenin oncogene has been known to increase the malignancy and metastasis of human breast cancers in vivo, the above findings could provide an effective therapy and/or anti-cancer drug for the prevention of cancer invasion and progression.

### EXAMPLE 3

### Ex-Vivo Transduction and Gene Silencing

Partial human immunodeficiency virus-1 (HIV-1) genome sequence from +1760 to +3196 bases was cloned into pCR2.1 plasmid vector (Invitrogen) for the preparation of a cDNA-aRNA hybrid probe homologous to HIV-1 gag-pro-pol genes. Since the pCR2.1 plasmid contains a T7 promoter in front of its antisense clone site, the aRNA portion of the cDNA-aRNA hybrid construct can be directly amplified in an in-vitro transcription reaction (20 μl), comprising 2 μl of 10×RT&T buffer (400 mM Tris-HCl, pH 8.3 at 25° C., 300 mM KCl, 80 mM MgCl₂, 2M betaine, 100 mM DTE and 20 mM spermidine), rNTPs (4 mM each for ATP GTP, CTP and UTP), T7 RNA polymerase (200U), RNase inhibitors (10U) and the above pCR2.1 plasmid (10 pg). The reaction was performed at 37° C. for two hours and then reverse transcription (40 μl) was continuously performed in the same tube by adding 2 μl of 10×RT&T buffer, dNTPs (4 mM each for dGTP, dCTP, dTTP and 2 mM each for dATP and dITP), MMLV reverse transcriptase (30U) and 1 μM sense primer 5′-dGGATGICIGI CICCTTGTTG GTCC-3′ (SEQ ID.3). The reaction was further incubated at 37° C. for two hours, so as to provide about 30 μg cDNA-aRNA hybrid construct for transfection.

Above HIV-1 cDNA-aRNA hybrid probe (10 μg) was dissolved in 200 mM calcium phosphate and directly applied to 2 ml culture flask contain 50% confluency of CD4⁺ Tc lymphocytes. The Tc lymphocytes were extracted from patients and can be grown in human serum extracts with 100 μg/ml interleukin 2 (IL-2) for two weeks. After 96 hr incubation, the gene activity of HIV-1 genome was measured by Northern blotting and found to be almost completely shut down in the cDNA-aRNA hybrid transfection set (FIG. 3(a), lane 5; and FIG. 3(b), lanes 3′, 5′ & 6′). The blank control (FIG. 3(a), lane 2; and FIG. 3(b), lane 1′) and other construct transfection (FIG. 3(a), lanes 4 & 6; and FIG. 3(b), lanes 4′ & 7′) sets had no significant gene silencing effects. Unlike dsRNA treatment, the transfection of high concentrated cDNA-aRNA hybrids (250 nM; FIG. 3(b), lane 6′) did not cause any interferon-induced killing effects, because the house-keeping gene β-actin is normally expressed in all sets of transfected cells as well as non-transfected HIV-1-negative control (FIG. 3(a), lane 2; and FIG. 3(b), lane 1′) and -positive (FIG. 3(a), lane 3; and FIG. 3(b), lane 2′) control sets. The FIG. 3(a) showed the acute transfection results of HIV-1 cDNA-aRNA hybrids in one-week-infection patients, while the FIG. 3(b) showed the chronic transfection results of HIV-1 cDNA-aRNA hybrids in two-year-infection patients. Because the Northern blot method is able to detect HIV-1 gene transcript at the nanogram level, the above strong viral gene silencing effect actually demonstrates a very promising pharmaceutical and therapeutical use of this cDNA-aRNA hybrid construct as antiviral drugs and/or vaccines.

### EXAMPLE 4

### In-Vivo Transduction and Gene Silencing

Partial Mus musculus tyrosinase (tyr) sense DNA (sDNA) sequence (SEQ ID.4) purchased from a core facility (Invitrogen) was synthesized by a oilgonucleotide synthesizer machine. The complementary antisense RNA (aRNA) sequence (SEQ ID.5) was transcribed from a tyr-inserted pCR2.1 plasmid vector using T7 RNA polymerase activity. The synthesized sDNA was boiled at 94° C., 10 min in diethyl pyrocarbonate-treated H2O(˜pH 5.5) for partial depurine. Such depurine will introduce some mismatched base pairs in a cDNA-aRNA hybrid. Hybridization of the tyr cDNA and aRNA was accomplished by incubation of 200 μg of each sequence in a 20 mM Hepes buffer (pH 6.5) at 68° C. for over 10 min and then gradually cooling from 50° C. to 10° C. over a period of one hour. The final cDNA-aRNA product was stored in a −80 freezer before used.

The dorsal hairs of one-month-old W-9 black mice were stripped by wax. Four intra-cutaneous injections of the tyr cDNA-aRNA (25 μg for each injection) were applied by a 24 hr interval fashion for each injection. After a thirteen-day hair regrowth period, white hairs were observed only in the injected area of the cDNA-aRNA transfected mice, while those of the dsRNA transfected and blank control mice showed normal black colored hairs. Northern analysis of the tyr gene expression indicated a 76.1±5.3% reduction in the transfected skins of the cDNA-aRNA treated mice, but no such gene silencing effect was found in the dsRNA transfected and blank control mice.

The present invention has been described with reference to particular preferred embodiments; however, the scope of this invention is defined by the attached claims and should be constructed to include reasonable equivalents.

Defined in broad, the present invention is a method for inducing gene silencing effects using cDNA-aRNA hybrid constructs, comprising the steps of:

a. providing a plurality of DNA sequences, wherein said DNA sequences are homologous to a or a plurality of targeted intracellular messenger RNA sequences;

b. contacting said DNA sequences to a plurality of RNA sequences to form a plurality of DNA-RNA hybrids, wherein said RNA sequences are complementary to said DNA and intracellular messenger RNA sequences; and

c. transducing said DNA-RNA hybrids into a plurality of cells which are sensitive to RNA interference effects; and so as to provide a specific gene silencing effect to the targeted messenger RNAs within said cells.

Alternatively defined in broad, the present invention is a method for generating cDNA-aRNA hybrids for gene silencing, comprising the steps of:

a. providing: i) a solution comprising a nucleic acid template, ii) one or more primers sufficiently complementary to the sense conformation of the nucleic acid template, and iii) one or more promoter-linked primers sufficiently complementary to the antisense conformation of the nucleic acid template, and having an RNA promoter;

b. treating the nucleic acid template with one or more primers under conditions such that a first cDNA strand is synthesized;

c. treating the first cDNA strand with one or more promoter-linked primers under conditions such that a promoter-linked double-stranded nucleic acid is synthesized;

d. treating the promoter-linked double-stranded nucleic acid under conditions such that essentially aRNA fragments are synthesized; and e) treating aRNA fragments with one or more primers under conditions such that a cDNA-aRNA hybrids are synthesized.

Alternatively defined in detail, the present invention is a method of improved RNA-polymerase cycling reaction which amplifies a specific DNA-RNA hybrid construct for transducing biological gene silencing effects, comprising the steps of:

a. providing a plurality of nucleic acid sequences as an amplifiable gene template for following reactions;

b. denaturing and contacting said nucleic acid template with a plurality of primers and a plurality of promoter-linked primers, wherein said primers and promoter-linked primers are respectively complementary to the sense and antisense sequence conformation of said nucleic acid template;

c. permitting extension of said primers and promoter-linked primers to form a plurality of promoter-linked double-stranded nucleic acid sequences, wherein said promoter-linked double-stranded nucleic acid sequences are formed by either DNA-directed or RNA-directed DNA and/or RNA polymerases or the combination thereof;

d. permitting transcription of said promoter-linked double-stranded nucleic acid sequences to form a plurality of amplified RNA fragments, wherein said amplified RNA fragments are generated by extension of RNA polymerase activity through the promoter region of said promoter-linked double-stranded DNAs; and

e. contacting said amplified RNA fragments with said primer to form a plurality of DNA-RNA hybrid duplexes, wherein said DNA-RNA hybrid duplexes are formed by reverse transcription of said amplified RNA fragments with the extension of said primer; so as to provide amplified cDNA-aRNA hybrids ready for inducing RNAi-related gene silencing effects.

Alternatively defined in broad, the present invention is a kit for inducing gene silencing effects using cDNA-aRNA hybrid constructs, comprising the components of:

a. a plurality of cDNA-aRNA hybrid constructs, wherein the cDNA portion of said cDNA-aRNA hybrid constructs are homologous to a or a plurality of targeted intracellular messenger RNA sequences; and

b. a plurality of transfection reagents, wherein said transfection reagents can deliver said cDNA-aRNA hybrid constructs into a plurality of targeted cells; and so as to provide a specific gene silencing effect to the targeted messenger RNAs within said cells.

It is understood that the examples and embodiments described herein are for illustrative purposes only and that various modifications or changes in light thereof will be suggested to persons skilled in the art, and are to be included within the spirit and purview of the invention as set forth in the appended claims. All publications and patents cited herein are incorporated herein by reference in their entirety for all purposes.

What we claim is:

1. A method for inducing gene silencing effects using sense DNA and antisense RNA (cDNA-aRNA) hybrids, comprising the steps of:
a. providing a plurality of DNA sequences, wherein said DNA sequences are homologous to a or a plurality of targeted intracellular messenger RNA sequences;
b. contacting said DNA sequences to a plurality of RNA sequences to form a plurality of DNA-RNA hybrids, wherein said RNA sequences are complementary to said DNA and intracellular messenger RNA sequences; and
c. transducing said DNA-RNA hybrids into a plurality of cells which are sensitive to RNA interference effects; and so as to provide a specific gene silencing effect to the targeted messenger RNAs within said cells.

2. The method as defined in claim 1, further comprising the generation of said DNA sequences by a machine selected from the group consisting of oilgonucleotide synthesizer, thermocycler and isothermal incubator.

3. The method as defined in claim 1, further comprising the generation of said DNA sequences from a plurality of template vectors by enzymatic methods selected from the group consisting of reverse transcription, polymerase chain reaction, nucleic acid sequence based amplification, and RNA-polymerase cycling reaction.

4. The method as defined in claim 3, wherein said template vectors are nucleic acid sequences containing said DNA sequences in the form of single-stranded, double-stranded, linear and/or circular structures.

5. The method as defined in claim 1, wherein said DNA sequences are completely homologous to said intracellular messenger RNA sequences.

6. The method as defined in claim 1, wherein said DNA sequences are partially homologous to said intracellular messenger RNA sequences.

7. The method as defined in claim 6, wherein said DNA sequences contain a plurality of nucleotide analogs selected from the group consisting of inosine, xanthine, hypoxanthine and their derivative analogs.

8. The method as defined in claim 1, further comprising the generation of said RNA sequences by a machine selected from the group consisting of oilgonucleotide synthesizer, thermocycler and isothermal incubator.

9. The method as defined in claim 1, further comprising the generation of said RNA sequences from a plurality of template vectors by enzymatic methods selected from the group consisting of in-vitro transcription, aRNA amplification, nucleic acid sequence based amplification, and RNA-polymerase cycling reaction.

10. The method as defined in claim 9, wherein said template vectors are nucleic acid sequences containing said RNA sequences in the form of single-stranded, double-stranded, linear and/or circular structures.

11. The method as defined in claim 1, wherein said RNA sequences are completely complementary to said DNA sequences.

12. The method as defined in claim 1, wherein said RNA sequences are partially complementary to said DNA sequences.

13. The method as defined in claim 1, further comprising the hybridization of said DNA and RNA sequences to form duplex sequences.

14. The method as defined in claim 13, wherein said DNA and RNA sequences are hybridized in a Hepes-containing buffer at about 68° C. for more than 10 minuets.

15. The method as defined in claim 14, wherein said Hepes-containing buffer is 20 mM HEPES solution.

16. The method as defined in claim 13, wherein said duplex sequences are completely matched.

17. The method as defined in claim 13, wherein said duplex sequences are partially matched.

18. The method as defined in claim 17, wherein said duplex sequences contain mis-matched base pairs selected from the group consisting of inosine, xanthine, hypoxanthine and their derivative analogs.

19. The method as defined in claim 1, further comprising the transduction of said DNA-RNA hybrids into said cells by a method selected from the group consisting of micro-injection, liposomal transfection, calcium phosphate transfection, chemical transformation, and electroporesis.

20. The method as defined in claim 1, wherein said cells contain RNA-directed endoribonucleases (RDE) for inducing RNA interference effects by the derivatives of said RNA sequences in said cells.

21. The method as defined in claim 20, wherein said RNA-directed endoribonuclease are naturally possessed by the said cells or introduced recombinantly into the said cells.

22. A method for generating cDNA-aRNA hybrids for gene silencing, comprising the steps of:
a. providing: i) a solution comprising a nucleic acid template, ii) one or more primers sufficiently complementary to the sense conformation of the nucleic acid template, and iii) one or more promoter-linked primers sufficiently complementary to the antisense conformation of the nucleic acid template, and having an RNA promoter;
b. treating the nucleic acid template with one or more primers under conditions such that a first cDNA strand is synthesized;
c. treating the first cDNA strand with one or more promoter-linked primers under conditions such that a plurality of promoter-linked double-stranded nucleic acid templates are synthesized;
d. treating the promoter-linked double-stranded nucleic acid templates under conditions such that essentially aRNA fragments are synthesized; and
e. treating aRNA fragments with one or more primers under conditions such that a plurality of cDNA-aRNA hybrids are synthesized.

23. The methods as defined in claim 22, can comprise the step of repeating steps b) through e) for a sufficient number of cycles to obtain a desired amount of amplified hybrid product.

24. The methods as defined in claim 22, the treating step in step b) can comprise heating the solution at a temperature above 90° C. to provide denatured nucleic acids.

25. The methods as defined in claim 22, the treating step in step c) can comprise treating the first cDNA strand with one or more promoter-linked primers at a temperature ranging from about 37° C. to about 72° C.

26. The methods as defined in claim 22, the treating step in step c) can also comprise treating the cDNA strand with one or more promoter-linked primers in the presence of a plurality of polymerases.

27. The methods as defined in claim 26, wherein said polymerase is selected from the group consisting of DNA-dependent DNA polymerases, RNA-dependent DNA polymerases, RNA polymerases, Taq-like DNA polymerase, Tth-like DNA polymerase, C. therm. polymerase, viral replicases, and combinations thereof. The viral replicases can be selected from the group consisting of avian myeloblastosis (AMV) reverse transcriptase and Moloney murine leukemia virus (MMLV) reverse transcriptase. In particular, the AMV reverse transcriptase does not have RNase H activity.

28. The methods as defined in claim 22, the treating step in step d) further comprise treating the promoter-linked double-stranded nucleic acid with an enzyme having transcriptase activity at about 37° C.

29. The methods as defined in claim 28, wherein said enzyme having transcriptase activity can be selected from the group consisting of RNA polymerases and viral replicases.

30. The methods as defined in claim 29, wherein said RNA polymerases can be selected from the group consisting of T3 RNA polymerase, T7 RNA polymerase, SP6 RNA polymerase, and M13 RNA polymerase.

31. The methods as defined in claim 29, wherein said viral replicases can be selected from the group consisting of avian myeloblastosis (AMV) reverse transcriptase and Moloney murine leukemia virus (MMLV) reverse transcriptase. In particular, the AMV reverse transcriptase does not have RNase H activity.

32. The methods as defined in claim 22, wherein said primers are complementary to the 3′-ends of the antisense conformation of the nucleic acid template, comprising a sequence-specific primer homologous to the targeted gene transcript.

33. The methods as defined in claim 22, wherein said promoter-linked primers are complementary to the 3′-ends of the sense conformation of the nucleic acid template, comprising a sequence-specific primer complementary to the targeted gene transcript, such as T7 promoter-linked poly(dT) primers.

34. The methods as defined in claim 22, wherein said promoter-linked double-stranded nucleic acid template can be selected from the group consisting of linear and circular promoter-containing double-stranded DNAs or promoter-linked partial single-stranded DNAs.

35. The methods as defined in claim 22, the treating step in step e) comprises treating aRNA fragments with one or more primers at a temperature ranging from about 37° C. to about 70° C.

36. The methods as defined in claim 22, further comprising the step of incorporating one or more nucleotide analogs into the cDNA portion of the cDNA-aRNA hybrid to facilitate the onset of RNAi-related effects.

37. The methods as defined in claim 36, wherein said nucleotide analog is selected from the group consisting of inosine, xanthine, hypoxanthine and their derivative nucleotides.

38. The methods as defined in claim 22, further comprising the step of contacting cDNA-aRNA hybrids with a reagent for gene silencing transfections.

39. The methods as defined in claim 38, wherein said reagent can be selected from the group consisting of electroporesis media, chemical transduction reagents and liposomal transfection reagents.

40. A method of improved RNA-polymerase cycling reaction which amplifies a specific DNA-RNA hybrid construct for transducing biological gene silencing effects, comprising the steps of:
a. providing a plurality of nucleic acid sequences as an amplifiable gene template for following reactions;
b. denaturing and contacting said nucleic acid template with a plurality of primers and a plurality of promoter-linked primers, wherein said primers and promoter-linked primers are respectively complementary to the sense and antisense sequence conformation of said nucleic acid template;
c. permitting extension of said primers and promoter-linked primers to form a plurality of promoter-linked double-stranded nucleic acid sequences, wherein said promoter-linked double-stranded nucleic acid sequences are formed by either DNA-directed or RNA-directed DNA and/or RNA polymerases or the combination thereof;
d. permitting transcription of said promoter-linked double-stranded nucleic acid sequences to form a plurality of amplified RNA fragments, wherein said amplified RNA fragments are generated by extension of RNA polymerase activity through the promoter region of said promoter-linked double-stranded DNAs; and
e. contacting said amplified RNA fragments with said primer to form a plurality of DNA-RNA hybrid duplexes, wherein said DNA-RNA hybrid duplexes are formed by reverse transcription of said amplified RNA fragments with the extension of said primer; so as to provide amplified cDNA-aRNA hybrids for gene silencing effects.

41. The method as defined in claim 40, further comprising repeated steps (b) through (e) on said amplified DNA-RNA hybrids at least one time.

42. The method as defined in claim 40, further comprising the step of generating and/or incorporating a plurality of nucleotide analogs into the cDNA part of said amplified cDNA-aRNA hybrids in the step (e) for the increase of onset of gene silencing effects.

43. The method as defined in claim 42. wherein said nucleotide analog is generated by deaminase.

44. The method as defined in claim 42. wherein said nucleotide analog is generated by chemical treatments.

45. The method as defined in claim 42. wherein said nucleotide analog is selected from the group consisting of inosine, xanthine, hypoxanthine and their derivative analogs.

46. The method as defined in claim 40, further comprising the step of mixing said amplified cDNA-aRNA hybrids with a plurality of reagents after step (e) for gene silencing or knockout transfection.

47. The methods as defined in claim 46, wherein said reagent can be selected from the group consisting of electroporesis media, chemical transduction reagents and liposomal transfection reagents.

48. The method as defined in claim 40, wherein said nucleic acid template need to be denatured at temperature ranged from about 90° C. to about 100° C. in the step (b).

49. The method as defined in claim 40, wherein said promoter-linked double-stranded nucleic acid sequences are selected from the group consisting of linear and circular forms of promoter-linked double-stranded DNAs and promoter-linked single-stranded DNAs.

50. The method as defined in claim 40, wherein said DNA-directed or RNA-directed DNA and/or RNA polymerases are enzyme activities selected from the group consisting of AMV reverse transcriptase without RNase H activity/Taq-like DNA polymerase mixtures, thermostable MMLV reverse transcriptase/ Taq-like DNA polymerase mixtures, Tth-like DNA polymerases with reverse transcription activity, C. therm. polymerases, and RNA polymerase/replicase mixture.

51. The method as defined in claim 50, wherein said enzyme activities are performed at temperature ranged from about 37° C. to about 70° C.

52. The method as defined in claim 40, wherein said primers are complementary to the 3′-ends of the antisense conformation part of said nucleic acid template.

53. The method as defined in claim 40, wherein said promoter-linked primers are complementary to the 3′-ends of the sense conformation part of said nucleic acid template.

54. The method as defined in claim 40, wherein said RNA polymerase or replicase activity is an enzyme activity selected from the group consisting of T3, T7, SP6 and M13 RNA polymerase and viral replicases.

55. The method as defined in claim 54, wherein said RNA polymerase activity is performed at temperature ranged about 37° C.

56. The method as defined in claim 40, wherein said cDNA-aRNA hybrid duplexes are formed by the group consisting of AMV reverse transcriptases without RNase H activity, thermostable MMLV reverse transcriptases, Tth-like DNA polymerases with reverse transcription activity and C. therm. polymerases.

57. The method as defined in claim 56, wherein said cDNA-aRNA hybrid duplexes are form at temperature ranged from about 37° C. to about 70° C.

58. The method as defined in claim 40, wherein said cDNA-aRNA hybrid duplexes are selected from the group consisting of pathogenic nucleic acids, viral genes, mutated genes, oncogenes, known functional genes or unknown functional nucleic acid sequences.

59. A kit for inducing gene silencing effects using cDNA-aRNA hybrids, comprising the components of:
a. a plurality of cDNA-aRNA hybrid constructs, wherein the cDNA portion of said cDNA-aRNA hybrid constructs are homologous to a or a plurality of targeted intracellular messenger RNA sequences; and
b. a plurality of transfection reagents, wherein said transfection reagents can deliver said cDNA-aRNA hybrid constructs into a plurality of targeted cells; and so as to provide a specific gene silencing effect to the targeted messenger RNAs within said cells.

60. The kit as defined in claim 59, further comprising the generation of said cDNA-aRNA hybrid constructs by a machine selected from the group consisting of oilgonucleotide synthesizer, thermocycler and isothermal incubator.

61. The kit as defined in claim 59, further comprising the generation of said cDNA-aRNA hybrid constructs from a plurality of template vectors by enzymatic methods selected from the group consisting of reverse transcription, polymerase chain reaction, nucleic acid sequence based amplification, and RNA-polymerase cycling reaction.

62. The kit as defined in claim 61, wherein said cDNA-aRNA hybrid constructs are generated by RNA-polymerase cycling reaction (RNA-PCR).

63. The kit as defined in claim 61, wherein said template vectors are nucleic acid sequences containing the cDNA portion of said cDNA-aRNA hybrid constructs in the form of single-stranded, double-stranded, linear and/or circular structures.

64. The kit as defined in claim 59, wherein said cDNA-aRNA hybrid constructs are completely homologous to said intracellular messenger RNA sequences.

65. The kit as defined in claim 59, wherein said cDNA-aRNA hybrid constructs are partially homologous to said intracellular messenger RNA sequences.

66. The kit as defined in claim 59, wherein said cDNA-aRNA hybrid constructs contain a plurality of nucleotide analogs selected from the group consisting of inosine, xanthine, hypoxanthine and their derivatives.

67. The kit as defined in claim 59, further comprising the hybridization of a plurality of DNA and RNA sequences to form said cDNA-aRNA hybrid constructs.

68. The kit as defined in claim 67, wherein said DNA and RNA sequences are completely complementary to each other.

69. The kit as defined in claim 67, wherein said DNA and RNA sequences are partially complementary to each other.

70. The kit as defined in claim 67, wherein said DNA and RNA sequences contain mis-matched base pairs selected from the group consisting of inosine, xanthine, hypoxanthine and their derivatives.

71. The kit as defined in claim 59, wherein said transfection reagent can be selected from the group consisting of electroporesis media, chemical transduction reagents and liposomal transfection reagents.

72. The kit as defined in claim 59, further comprising the transduction of said cDNA-aRNA hybrids into said targeted cells by a delivery method selected from the group consisting of micro-injection, liposomal transfection, calcium phosphate transfection, chemical transformation, vector penetration and electroporesis.

73. The kit as defined in claim 59, wherein said targeted cells contain RNA-directed endoribonucleases for inducing RNA interference effects by the derivatives of said cDNA-aRNA hybrids in said cells.

74. A composition comprises a plurality of DNA-RNA hybrids, wherein said DNA-RNA hybrids are capable of inducing sequence-specific gene silencing or knockout effects in cells or organisms.

75. The composition as defined in claim 74, wherein said cells are of high vertebrate origins.

76. The cells as defined in claim 75, wherein said cells are selected from a group of cultured tissue cells comprising mouse, rat, rabbit, canine, chicken and human cells.

77. The composition as defined in claim 74, wherein said organisms are mammalian origins.

78. The organisms as defined in claim 77, wherein said organisms are selected from a group of animals comprising mouse, rat, rabbit, canine and human beings.

79. A therapeutic strategy, measurement or treatment for human diseases using DNA-RNA hybrid construct as claimed in claim 59 and/or 74.

80. The therapeutic treatment as defined in claim 79 is used for treating human tumors and cancers.

81. The therapeutic treatment as defined in claim 79 is used for treating viral infections.

82. The virus infections as defined in claim 81 are selected from a group of HIV, HCV, common cold Rhinovirus, Herpes virus, CMV, Ebola virus, oncogenic retrovirus, and other human-disease-inducing and/or -associated virus infections.

83. A strategy for gene-based research and therapy using cDNA-RNA hybrid transfection against oncogene expression.

84. A strategy for gene-based therapy using cDNA-RNA hybrid transfection against human immunodeficiency virus (HIV) gene expression and/or infection.

85. A process uses a composition as defined in claim 74.

86. The said process in claim 85 is useful for gene function analysis, drug discovery, drug target identification and drug target validation.

87. A cell line derived from application of a composition as defined in claim 74 or utilizes a process as defined in claim 85.

88. An organism derived from application of a composition as defined in claim 74 or utilizes a process as defined in claim 85.

89. Information and informational data generated from a process as claimed in claim 85, or derived from phenotypical and/or genotypical observations of a cell line of claim 87 and/or an organism of claim 88.

90. A gene sequence useful for therapeutic intervention or treatment and diagnostics and prognostics as revealed with the application a composition as claimed in claim 74 and/or the use of a process as claimed in claim 85.

91. A protein product derived from the said sequence in claim 90 is useful for therapeutic intervention or treatment and diagnostics and prognostics.

92. A drug target useful for screening therapeutic small molecule chemical drugs and antibody drugs and as diagnostic and prognostic marker as revealed with the application a composition as claimed in claim 74 and/or the use of a process as claimed in claim 85.

93. A therapeutic drug for human and animal diseases derived from using a method of claim 1, a composition of claim 74, a process of claim 85, a cell line of claim 87, an organism of claim 88, informational data of claim 89, a gene sequence of claim 90, a protein of claim 91 or a drug target of claim 92.

94. A diagnostic or prognostic marker for human and animal diseases derived from using a method of claim 1, a composition of claim 74, a process of claim 85, a cell line of claim 87, an organism of claim 88, informational data of claim 89, a gene sequence of claim 90, a protein of claim 91 or a drug target of claim 92.
